(19) United States
(12) Patent Application Publication
(10) Pub. No.: US 2025/0277182 A1
(43) Pub. Date: Sep. 4, 2025

(54) THREE-DIMENSIONAL CELL CULTURE MONITORING

(71) Applicant: The Texas A&M University System, College Station, TX (US)

(72) Inventors: Oscar R. Benavides, Bryan, TX (US); Alexandra J. Walsh, College Station, TX (US); Holly C. Gibbs, Bryan, TX (US); Kristen C. Maitland, College Station, TX (US)

(21) Appl. No.: 19/070,224

(22) Filed: Mar. 4, 2025

Related U.S. Application Data
(60) Provisional application No. 63/561,267, filed on Mar. 4, 2024.

(51) Int. Cl. C12M 1/34; C12M 1/00; C12M 3/06

(52) U.S. Cl. CPC C12M 41/46; C12M 23/16; C12M 31/10

(57) ABSTRACT

Three-dimensional cell cultures, such as microcarrier-based cell cultures, can be visualized using light sheet microscopy (LSM) with elastic-scattering or fluorescence contrast to optically section a culture sample into a sequence of 2D images, from which a 3D image can then be reconstructed. Further analysis of the 3D image enables measuring various cell culture parameters for quantitative monitoring. In various embodiments, elastic-scattering LSM in conjunction with flow-based translation of the culture sample through the light sheet moreover facilitates non-destructive, online monitoring of the cell culture.

### CROSS-REFERENCE TO RELATED APPLICATION

This application claims priority and the benefit of U.S. Provisional Patent Application No. 63/561,267, filed on Mar. 4, 2024, which is hereby incorporated herein by reference in its entirety.

### BACKGROUND

The adoption of cell-based therapies (or “cytotherapies”) into clinical practice entails a growing demand for cell expansion (that is, the process of growing and multiplying cells in culture under controlled conditions) at large scale. For example, stem-cell-based therapies, which show great promise in combating chronic diseases, typically require billons of cells per indication per year. Conventional two-dimensional (2D) monolayer cell cultures are significantly outperformed, in generating cells in high numbers, by three-dimensional (3D) cell cultures in bioreactors, and in particular by microcarrier-based cell cultures, where cells are grown on the surface of microcarriers suspended in a liquid medium. However, 3D cell cultures pose challenges to in-line or on-line monitoring of cell numbers, cell morphology, and overall culture health. Standard visualization-based cell culture monitoring methods that have been successfully used with 2D cell cultures are not readily translatable to 3D cell cultures. For instance, when wide-field microscopy, which has been a mainstay in 2D cell culture for decades, is applied to 3D microcarrier-based cultures, it produces a single, blurred image of only the proximal microcarrier hemisphere, and does not permit visualization of the entire microcarrier surface area, limiting quantitative analysis of the cell culture. Further, conventional cell density and viability measurements are typically performed using trypan blue dye exclusion or live/dead fluorescence assays, both of which require the uptake of an exogenous compound, and are destructive to cells, prohibiting live cell imaging. Accordingly, the development of novel cell expansion strategies using 3D cell cultures calls for the simultaneous advancement of analytical tools to study and monitor these cultures, including nondestructive in-line or on-line methods for visualizing and characterizing cells.

### BRIEF DESCRIPTION OF THE DRAWINGS

Disclosed herein are systems and methods for monitoring 3D cell culture using light sheet microscopy.

FIG. 1 conceptually depicts an example system for the flow-based on-line monitoring of 3D cell culture, in accordance with various embodiments.

FIG. 2A is a schematic diagram of an example light sheet microscopy system enabling both elastic-scattering light sheet microscopy and light sheet fluorescence microscopy, and FIGS. 2B and 2C are schematic diagrams of alternative configurations of the detection path of the system for multi-channel detection, in accordance with various embodiments.

FIG. 3 is an array of example 2D elastic-scattering images of a microcarrier-based cell culture sample taken with the system of FIG. 2A for twelve different polarization state configurations, illustrating the dependence of image contrast on polarization.

FIGS. 4A-4D shows example 2D images of a microcarrier-based cell culture sample taken with the system of FIG. 2A using various imaging modalities.

FIG. 5 shows an example sequence of 2D elastic-scattering images of a microcarrier-based cell culture sample taken with the system of FIG. 2A using flow-based translation of the sample relative to the light sheet, in accordance with various embodiments.

FIGS. 6A-6G illustrate the reconstruction of a 3D volumetric image of a sample from a sequence of 2D images of slices sample, in accordance with various embodiments.

FIGS. 7A and 7B are projections of example 3D volumetric images of cells cultured on a microcarrier as reconstructed from elastic-scattering light sheet microscopy data and light sheet fluorescence microscopy data, respectively.

FIG. 8 is a block diagram of an example machine suitable for controlling the operation of the systems of FIGS. 1 and 2 and/or implementing functionality for processing and analyzing the elastic-scattering and/or fluorescence images, in accordance with various embodiments.

### DESCRIPTION

Disclosed herein are systems and methods for performing quantitative visualization-based monitoring of 3D cell cultures using light sheet microscopy (LSM) (also sometimes referred to as selective plane illumination microscopy (SPIM)). Various described aspects pertain to an LSM microscopy system with elastic-scattering-based and/or fluorescence-based imaging modalities, a fluidic sampling system configured to deliver 3D cell culture samples taken from a bioreactor or other cell culture system to the microscopy system for flow-based imaging, and an image processing and analysis system for processing the acquired image data to generate 3D visualizations of the samples and, in many cases, automatically determine quantitative cell culture parameters.

In LSM, only a thin, planar slice or “sheet” (that is, a volume whose thickness is very small as compared with its lateral dimensions) within the sample is illuminated, and the detection optics are focused on the illuminated plane, such that the contribution of out-of-focus light to image formation is inherently minimized, resulting in a high-resolution image of a single slice within the sample. This stands in contrast to wide-field microscopy, which involves illuminating and capturing light from the entire sample, and as a result produces blurred images of 3D objects because the out-of-focus light does contribute to image formation, obscuring high-frequency spatial features. While LSM, unlike wide-field microscopy, does not image the entire 3D sample at once, it allows optically sectioning the sample by translating the sample relative to (and through) the thin illumination light sheet to acquire a sequence, or stack, of 2D images, from which a 3D volumetric image of the sample can be reconstructed. This capability is leveraged, in accordance with various embodiments, for the high-resolution visualization of 3D cell cultures, in particular cell cultures grown in suspension, such as suspension cell cultures (where individual suspension-adapted cells grow floating in liquid culture medium), aggregate cell cultures (where cells clump together to form multicellular aggregates suspended in liquid culture medium), or microcarrier-based cell cultures (where cells adhere to and grow on the surface of microcarriers suspended in liquid culture medium). Some embodiments utilize the flow of a sample of the cell culture suspension through the light sheet to achieve the relative motion between sample and light sheet that is needed for optical sectioning. In this case, the ratio of flow velocity and image acquisition rate can be selected to achieve a desired predetermined thickness of the imaged sample slices.

In various embodiments, the microscopy system supports an imaging modality herein referred to as elastic-scattering LSM (esLSM), which involves illuminating a sheet within the sample, usually with visible light (e.g., at 633 nm), and detecting elastically scattered light (that is, light at the same wavelength) to generate contrast for image formation. Beneficially, using elastic-scattering contrast provides a label-free and thus non-destructive method of imaging, enabling live cell imaging, which in turn facilitates in-line and on-line cell culture monitoring. In-line monitoring refers to the real-time measurement of cell-culture parameters directly in the cell culture system (e.g., bioreactor) where the cells are grown, whereas on-line monitoring refers to the near real-time monitoring of a sample that is removed from the cell culture system for imaging and subsequently returned to the cell culture system without disruption of the culture process, usually in an automated manner. In some embodiments, a closed-loop fluidic sampling system is employed in on-line monitoring to transport the cell culture sample from the cell culture system to the microscopy system and back.

In some embodiments, the microscopy system provides, alternatively or additionally to esLSM, an imaging modality known as light sheet fluorescence microscopy (LSMF). In LSM, fluorescence contrast for imaging is achieved by illuminating a suitably labeled (or, in some cases, intrinsically fluorescent) sample at an associated excitation wavelength and measuring the emitted fluorescence light. LSFM has been the gold standard in cell culture monitoring, but is often destructive and thus in practice limited to off-line monitoring. In various beneficial embodiments, the microscopy system provides both esLSM and LSFM capabilities, facilitating a direct comparison of elastic-scattering contrast images with fluorescence contrast images of the same sample, which can be used to calibrate the image processing and analysis of esLSM image data based on reference LSFM data.

In some embodiments, to enable the use of elastic-scattering contrast and/or optimize imaging conditions, the refractive indices along the optical path between illumination and detection objective lenses of the microscopy system are substantially matched (e.g., differ from each by no more than 0.03). For example, if the sample, which is generally an aqueous suspension, is imaged through a sample chamber or section of tubing, the walls of the sample chamber or the tubing may be of a material selected to have a refractive index close to the refractive index of water, which is about 1.33; suitable materials include polymers with a refractive index between 1.32 and 1.35 in the visible regime, such as fluorinated ethylene propylene (FEP), which has a refractive index of 1.34. Further, the space between the microscope objective lenses used to focus the illumination light and capture the elastically scattered light may be filled with an index-matched material, such as water or an ultrasound gel having an index between about 1.33 and 1.35. In addition, for microcarrier-based cell cultures, the microcarriers may be chosen based in part on their refractive index to provide an index contrast to the cells sufficient to allow distinguishing between cells and microcarriers, and to provide an index contrast to water that is high enough to enable detection of the microcarriers, yet low enough to avoid excessive scattering that would interfere with imaging cells through the microcarriers; an example of a suitable microcarrier material is gelatin methacryloyl (gelMA), whose refractive index is about 1.35. In some embodiments, the microscopy system includes one or more polarizers in the path of the illumination light (herein also the “illumination path”) and one or more polarization analyzers in the path of the elastically scattered light (herein also the “detection path”) to facilitate further improving the image contrast. The microscopy system may also include a zoom lens for variable field of view and magnification to facilitate optimizing imaging across spatial scales.

Once a 3D volumetric image of the sample has been reconstructed from a sequence of 2D images, the reconstructed 3D image can be further analyzed, by suitable computer algorithms of the image processing and analysis system, to determine various parameters of the cell culture, such as, e.g., cell number, cell density, cell size distribution or average, or cell morphology distribution or average. When applied to a microcarrier-based cell culture, parameters of interest may also include the number of microcarriers and the average number of cells on each microcarrier. In some embodiments, image analysis is based on explicit rules. For example, intensity-based thresholding may be used to segment the image into regions of high-intensity image voxels associated with biomass (such as cells) and regions of lower-intensity image voxels associated with microcarriers and the surrounding aqueous environment. The number of voxels included in the biomass region may be summed to give a measure of cell volume (e.g., in μm³). The volume of the microcarriers can be measured in the same manner using suitably thresholded low-intensity voxels. The percentage of covered microcarrier surface area can be quantified as well based on this data. In some embodiments, instead of using rules-based analysis, the 3D reconstructed image is provided as input to a machine learning model to directly output cell culture parameters. The model may result, e.g., from supervised training on 3D images reconstructed from esLSM images, labeled with cell culture parameters derived from corresponding LSFM-based 3D images of the same respective samples.

Quantitatively characterizing the cell culture in terms of parameters such as, e.g., the average number of cells on each microcarrier and/or the average cell shape and size, among others, allows assessing and monitoring the health and proliferation of the cell cultures. Further, automating this quantitative image analysis permits higher-throughput and more robust evaluation than subjective human evaluation of cell number and morphology. As a result, esLSM-based, on-line imaging in conjunction with automated image processing and analysis provides the potential to serve as a Process Analytical Technology (PAT) for the real-time or near real-time monitoring of cell cultures in large-scale manufacturing contexts (e.g., the manufacture of cell-based therapies), increasing understanding of the cell culture process and facilitating control of critical process parameters that influence the quality and safety of the cell product.

Following this overview of various aspects and features of LSM-based cell culture monitoring, certain embodiments and examples will now be described in more detail with reference to the drawings. It is to be understood that embodiments of the disclosed subject matter are not limited to the specific combinations of features illustrated, but are intended to encompass other combinations of features that may occur to a person of ordinary skill in the art given the benefit of the instant specification. For example, although the following discussion focuses on flow-based, on-line monitoring of microcarrier-based cell cultures, various imaging and processing techniques described are also applicable to in-line or off-line monitoring and/or to other types of cell cultures.

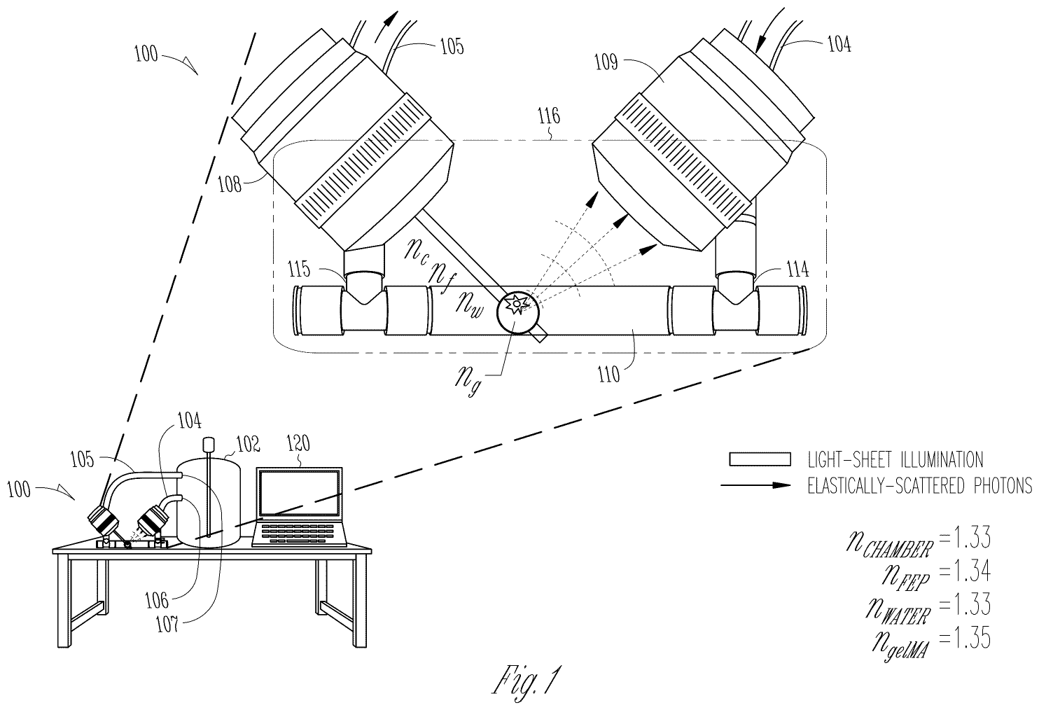

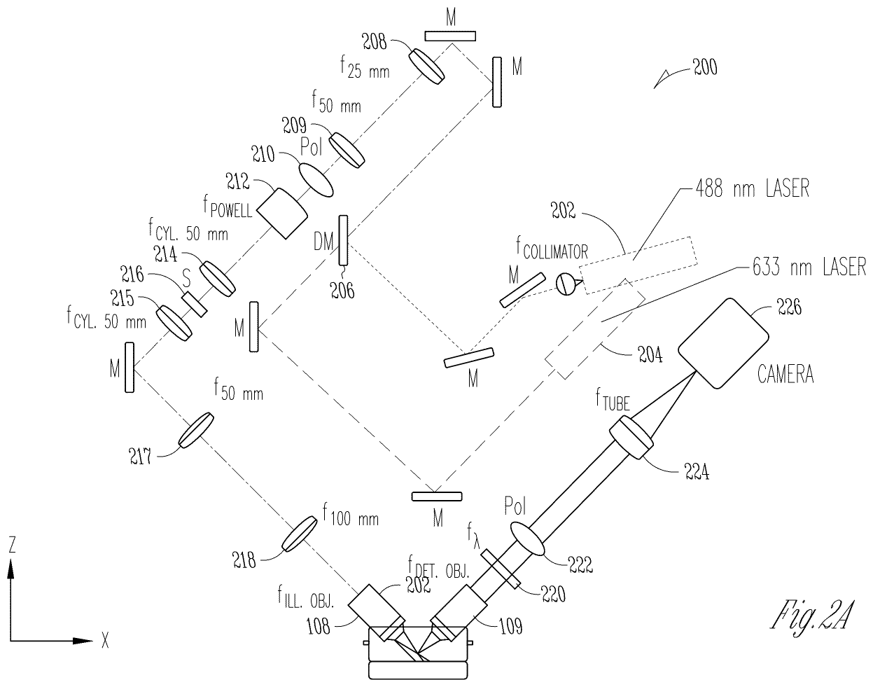

FIG. 1 conceptually depicts an example system 100 for the flow-based on-line monitoring of 3D cell culture, in accordance with various embodiments. The cell culture is grown in a bioreactor 102, such as, e.g., a rotating wall vessel bioreactor, vertical wheel bioreactor, stirred tank bioreactor, or rocking motion bioreactor. Various subsystems, including an LSM system, a fluidic sampling system, and a processing system including functionality for image processing and analysis, are configured to facilitate monitoring the cell culture. As part of the fluidic sampling system, a closed-loop sampling line, including tubing 104, 105 (having a diameter, e.g., in the range from 0.5 mm to 5 mm), is connected between two access ports 106, 107 of the bioreactor 102 (such that the sampling line and bioreactor 102 together form a closed fluidic loop), allowing sampling of the cell culture suspension from the bioreactor 102 and transport to the microscopy system and back to the bioreactor 102. The microscopy system itself is represented in FIG. 1 merely by the two microscope objectives—the illumination objective 108 and detection objective 109—that are used to focus illumination light into a light sheet and to collect elastically scattered light emitted from the illuminated sample slice, respectively; other components of the microscopy system are shown in FIG. 2A. The illumination and detection objectives 108, 109 are oriented perpendicularly (or substantially perpendicularly, e.g., with their optical axes enclosing an angle between 85° and 95°) to one another, which minimizes background illumination light as well as contributions of out-of-focus light in the detected image.

As depicted in FIG. 1, the sampling line may include a sample chamber 110 fixedly located within (although not itself forming part of) the microscopy system. The sample chamber 110 may, for example, be mounted to a sample stage of the microscopy system, or otherwise held in a fixed position and orientation relative to the microscope objectives 108, 109 such that the light sheet for LSM is created inside the sample chamber 110. The sample chamber 110 may be implemented, for example, by a fixed section of tubing or in a microfluid chip, with fluidic input and output ports coupled, e.g., via 90-degree connectors 114, 115 (such as elbow connectors or, as shown, T-connectors with one port closed) to the tubing 104, 105 leading to the access ports 106, 107 of the bioreactor 102. The tubing 104, 105, sample chamber 110, and connectors 114, 115 together form the closed-loop sampling line. In the depicted example, the illumination and detection objectives 108, 109 are placed above the sample. However, configurations in which either or both objectives 108, 109 are placed below the sample are also feasible. For example, in an inverted illumination configuration, the illumination objective 108 focuses light into the sample from below. It is also possible to use two illumination objectives placed on opposite sides of the sample chamber 110 (e.g., the depicted illumination objective 108 and a second illumination objective aligned along the same optical axis, but facing in the opposite direction) for dual-sided light sheet illumination.

To provide an “optical window” to image through, portions of the walls defining the sample chamber 110 that are within the path of the illumination and/or the scattered/fluorescence light, such as a tubing section implementing the chamber or a cover of a microfluidic chip defining the chamber, may be made from a material that is refractive-index-matched to water, such as FEP. In some embodiments, for simplicity, a single piece of tubing made of FEP or another suitable material is connected between the access ports 106, 107 of the bioreactor 102, with a section of the tubing serving as the sample chamber 110. Additionally, the space between the lenses of the illumination and detection objectives 108, 109 and the walls of the sample chamber 110 may be filled with an index-material (rather than air). For example, the objectives 108, 109 may have water-immersion lenses, used with both objectives 108, 109 and sample chamber 110 immersed in a water-filled container 116, as shown. Alternatively, the objectives 108, 109 may be optically coupled to the sample chamber 110 via an index-matching gel, e.g., hydrogel.

The fluidic sample system includes, in addition to the closed-loop sampling line, one or more associated pumps (not shown) that, when turned on, create hydraulic flow (that is, flow driven by pressure differences) that causes suspension to be continuously sampled from the bioreactor 102 and flown through the closed-loop sampling line, including through the sample chamber 110, where the imaging takes place. By virtue of the flow through the sample chamber, the sample is translated relative to the light sheet, resulting in optical sectioning of the sample. The pump(s) may be implemented, e.g., by syringe pumps, diaphragm pumps, peristaltic pumps, low-shear gear pumps, microfluidic pumps, etc. In various embodiments, the pump(s) are motorized and electronically controlled, allowing pump operation to be automated, e.g., to sample the cell culture at pre-programmed intervals or in response to defined sensed conditions, as well as to control the volumetric flow rate (e.g., measured in mL/s) and, thus, the flow velocity (e.g., measured in μm/s) through the sample chamber. This flow velocity, in conjunction with the image acquisition rate, or frame rate, of the microscopy system, determines the thickness of the sample slice imaged in each frame (assuming that the light sheet illuminates the entire sample slice). For a configuration in which the illumination light sheet is oriented at 45° relative to the direction of flow through the sample chamber, the slice thickness d_{slice} is computed from the flow velocity v_{flow} (e.g., measured in μm/s) and the image acquisition rate r_{acq} (e.g., measured in s⁻¹) according to: d_{slice}=v_{flow}/(\sqrt{2}ra_{cq}). For example, at a flow velocity of 45 μm/s and an image acquisition rate of 30 frames per second, the imaged sample slice will be about 1 μm thick.

The processing system 120 may be implemented by one or more general-purpose computers (e.g., personal computers or servers including one or more central processing units (CPUs) and/or graphic processing units (GPUs), along with volatile and/or non-volatile computer memory) executing suitable software programs. Alternatively or additionally, the processing system may include one or more special-purpose processors, such as, e.g., a microcontroller, digital signal processor (DSP), field-programmable gate array (FPGA), or application-specific integrated circuit (ASIC). In various embodiments, the processing system may be provided locally, in the vicinity of and directly connected to the fluidic sampling and/or microscopy systems, or remotely and communicatively coupled to the fluidic sampling and/or microscopy systems via a suitable network (e.g., a local-area network (LAN), wide-area network (WAN), or the internet). In general, the processing system implements the image processing and analysis system, that is, it receives 2D image data from the camera of the microscopy system, and processes the data to reconstruct 3D visualizations of culture samples as well as to quantitatively characterize the samples in terms of cell culture parameters. Additionally, the processing system may implement one or more controllers for the fluidic sampling system (in particular, the pump(s)) and microscopy system (e.g., the illumination light source and camera). For example, the processing system may turn the pump(s) of the fluidic sampling system on or off, as well as control the flow rate within the closed-loop sampling line, optionally coordinated with the operation of the camera (e.g., to adjust the flow rate based on a given image acquisition rate, or vice versa). In some embodiments, the system 100 includes flow sensors associated with the tubing 104, 105 to and from the microscopy system, e.g., implemented by pairs of a light-emitting diode (LED) on one side of the tubing and a photodetector (PD) on the other side of the tubing, that sense when a cell culture sample passes. The camera may be controlled based on signals from the flow sensor, e.g., mediated via a controller in the processing system, to start image acquisition when the sample passes the flow sensor in the tubing 104 from the bioreactor 102 to the sample chamber 110, and stop image acquisition when the sample passes the flow sensor in the tubing 105 from the sample chamber 110 back to the bioreactor 102.

Conventionally, cell enumeration to monitor the health of a cell culture has generally been performed by first physically sampling from the cell culture and then destructively imaging the sample. For example, monitoring microcarrier-based cultures has in the past involved trypsinization of the cells to remove them from the microcarrier surface, followed by the use of trypan blue dye exclusion or live/dead fluorescence assays, which both require the introduction of exogenous dyes that are destructive to the cell samples. By contrast, in accordance with various embodiments, e.g., using the above-described system 100, cells can be imaged without removing them permanently from the bioreactor culture. Instead, a closed-loop sampling line is used to temporarily take cell culture samples from the bioreactor for flow-based monitoring. This configuration maintains both sterilization and yield of the culture while enabling acquisition of high-resolution images.

Note that non-destructive imaging, as used in on-line monitoring, is generally performed using elastic-scattering contrast. For the purpose of fluorescence-based imaging, the system 100 can be straightforwardly modified to enable off-line monitoring. For instance, tubing 104 may be used to deliver a sample from the bioreactor 102 to a microfluidic device in the microscopy system, where fluorescent label can be introduced to the sample before the sample is fluorescently imaged with the microscopy system, (re-)configured to illuminate at an excitation wavelength and detect light at a different fluorescence wavelength. Following imaging, the sample can be discarded, rather than returned to the bioreactor. Off-line monitoring is also used to image the same sample using both esLSM and LSFM, which may serve to validate the image analysis performed based on the esLSM data using the LSFM data, or to acquire training data for training a machine-learning model to determine cell culture parameters from esLSM images, using LSFM-derived parameters as ground-truth labels.

FIG. 2A is a schematic diagram of an example light sheet microscopy system 200 enabling both esLSM and LSFM, in accordance with various embodiments. The fluorescence imaging modality may be provided alongside the elastic-scattering imaging capability to enable a direct comparison of elastic-scattering contrast images and data with the gold-standard fluorescence-based visualization of cell morphology.

In the illumination path, the system 200 includes two light sources 202, 204 for illumination: a 488 nm laser diode (e.g., Coherent OBIS 488 nm 40 mW), and a 633 nm laser diode (Coherent OBIS 633 nm 70 mW). The 488 nm light may be used, e.g., to excite the fluorescence of CellTrace Green (CTG), a cell-tracking dye commonly used to label cytoplasm for long-term cell proliferation and viability studies. The 633 nm light may be used, e.g., for elastic scattering, or to excite the fluorescence of DRAQ-5, a far-red fluorescent DNA stain. The beams from the two light sources 202, 204 are steered, with a suitable configuration of mirrors (M), onto a dichroic mirror 206 (DM) (e.g., Thorlabs DMLP505) that co-aligns them. Additional mirrors (M) are included in the system 200 to steer the coaligned beams to achieve a compact system configuration. The coaligned beams (which may be used together for esLSM/LSFM correlations studies, or separately to use only one of the imaging modalities) are magnified by passing through a pair of lenses 208, 209 with suitable focal lengths. For example, a two-fold magnification can be achieved with a 25 mm focal-length lens (e.g., Thorlabs AC254-025-A) and a 50 mm focal-length lens (e.g., Thorlabs AC254-050-a). Downstream of the pair of magnifying lenses 208, 209, a polarizer 210, such as a λ/2 plate (e.g., Newport 10RP42-1) or λ/4 plate (e.g., Edmund Optics 390-32), is placed in the optical path to create linear vertical, linear horizontal, right circular, or left circular illumination polarization states. Herein, horizontal polarization is polarization within the plane of the microscopy system, which is the plane of the figure (and, since light propagates as a transverse wave, perpendicular to the direction of light propagation), and vertical polarization is polarization normal to the plane of the figure (and thereby implicitly also perpendicular to the direction of propagation). A Powell lens 212 (e.g., Thorlabs LGL175) is used to generate a laser line with a flat-top, as opposed to gaussian, intensity profile.

The system 200 further includes illumination optics to create an illumination light sheet from the polarized, flat-top beam. Two cylindrical lenses 214, 215 (e.g., Thorlabs ACY254-050-A) collimate the beam along its fast axis (i.e., the axis along which the beam immediately after the Powell lens is diverging) and focus the beam along its slow axis (i.e., the axis along which the beam immediately after the Powell lens is collimated). A variable vertical slit 216 placed at the common focal plane of the cylindrical lenses 214, 215 is used to control the numerical aperture of the beam. The beam is then imaged, with a pair of lenses 217, 218 (e.g., a 50 mm focal-length lens and a 100 mm focal-length lens for a further two-fold magnification), onto the front focal plane of the illumination objective 108, which produces a focused illumination beam forming the illumination light sheet at a location in the sample chamber 110. The illumination objective 108 may be, e.g., a 10× 0.3 W NA objective (e.g., Olympus UMPLFLN 10×), and may be oriented at 45° with respect to the direction of flow through the sample chamber 110, which may be oriented parallel to the horizontal plane.

In the detection path, the emitted signal, e.g., elastically scattered light or fluorescence light, is collected by the detection objective 109, which is oriented orthogonal to the plane of the illumination light sheet. The detection objective 109 may be, e.g., a 20× 0.5 W NA objective (e.g., Olympus UMPLFLN 20×). In the infinity space of the detection objective 109, a long-pass emission filter 220 (e.g., using a Semrock BLP01-488R-25 filter) may be used, in fluorescence imaging, to block out the fluorescence excitation wavelength (e.g., 488 nm for fluorescence imaging of CTG-labeled cytoplasm, or 633 nm for fluorescence imaging of DRAQ-5-labeled cell nuclei. In elastic-scattering imaging, the filter 220 is removed to allow the scattered illumination light to be detected. To allow simultaneously detecting scattering at 633 nm and the 500+ nm fluorescence excited by 488 nm light and/or the 650+ nm fluorescence excited by 633 nm light, the filter is configured to block only the 488 nm excitation, e.g., using the configuration of FIGS. 2B or 2C. A second linear polarizer 222 (e.g., Thorlabs WP50L-UB) is placed in the infinity space of the detection objective 109 to change the detected polarization states. The field of view is then imaged with a tube lens 224 of suitable focal length onto the sensor of the camera 226. The tube lens 224 may, e.g., be a 150 mm focal-length lens (e.g., Thorlabs AC254-150-a). The camera may be, for example, a CMOS camera (e.g., pco.edge 5.5) or a charge coupled device (CCD) camera. With an example image sensor size of 2560×2160 pixels 6.5×6.5 μm² in size and the listed example tube lens 224 and detection objective 109, the field of view within the sample will be about 840×700 μm². This example microscopy system 200 can provide sub-cellular resolution of cells on microcarriers with both elastic-scattering contrast and fluorescence and contrast. In some embodiments, the tube lens 224 is a zoom lens, that is, a lens with variable focal length that allows adjusting the magnification and field of view.

In various embodiments, polarization control in the illumination and detection paths is used to improve or optimize the elastic-scattering image contrast. In the microscopy system 200, by selecting between a λ/4 waveplate and a λ/2 waveplate for the polarizer 210 and adjusting its orientation relative to incoming linearly polarized light, four polarization states—linear vertical, linear horizontal, right circular, and left circular—can be created. In the detection path, the linear polarizer 222, functioning as a polarization analyzer, creates one of three polarization states—linear vertical, linear horizontal, or no polarization. Together, the polarizers in the illumination and detection paths, thus, allow for twelve different system polarization state configurations.

FIGS. 2B and 2C are schematic diagrams of alternative configurations of the detection path of the microscopy system 200 that are suitable for multi-channel detection, in accordance with various embodiments. FIG. 2B illustrates the simultaneous, two-channel detection of elastically scattered light, e.g., at 633 nm, and fluorescence light excited, e.g., in CTG-labeled cells by 488 nm illumination (in the absence of DRAQ-5 labels). In this configuration, a dichroic mirror 230 separates out the 633 nm light from the 488 nm excitation light and the 500+ nm fluorescence light, and a long-pass filter 220 in the fluorescence path blocks the 488 nm excitation. A second dichroic mirror 232 realigns the transmitted fluorescence and scattering light before they are focused by the tube lens 224 onto different regions of the image sensor of the camera 226. FIG. 2C illustrates the simultaneous, three-channel detection of elastically scattered light, e.g., at 633 nm, fluorescence light excited, e.g., in CTG-labeled cells by 488 nm illumination, and fluorescence light excited, e.g., by DRAQ-labeled nuclei by 633 nm illumination. Here, the dichroic mirror 230 separates the 488 nm excitation light and corresponding 500+ nm fluorescence light from the 633 nm light and the 650+ nm fluorescence. In the 488 nm fluorescence path, the filter 220 blocks the 488 nm excitation. In the 633 nm path, an additional dichroic mirror 234 separates out the 650+ nm fluorescence light from the 633 nm scattered light. Optionally, the 650+ nm path may include a further filter to block any 633 nm light that has bled through or off the dichroic mirror 234. The dichroic mirror 232 realigns light at 500+ nm, 650+ nm, and 633 nm prior to focusing, by the tube lens 224, onto different regions of the image sensor of the camera 226.

As will be readily understood, the system 200 is but one example of a microscopy system suitable for esLSM and LSFM. Various modifications, including optical components with different parameters (e.g., focal length, magnification, etc.) or different combinations of optical components, which may retain some, but not necessarily all of the performance characteristics of the system 200, may occur to those of ordinary skill in the art.

Various aspects of image acquisition and processing are hereinafter discussed with reference to example image data. Unless otherwise noted, the cell cultures imaged in these examples are induced pluripotent stem cell (iPSC)-derived human mesenchymal stem cells (hereinafter iH-MSCs, in the scientific literature also alternatively abbreviated as iPSC-MSCs, hiPSC-MSCs, iMSC, or hiMSCs) grown on gelMA microcarriers about 120 μm in diameter. MSCs are attractive cytotherapeutic candidates due to their anti-inflammatory and immunomodulatory properties. Microcarriers can generally be made from a variety of materials, including solids such as polystyrene or glass, or hydrogels (of which gelMA is one example). Traditional solid microcarrier materials, however, have high refractive indices (e.g., ˜1.6 for polystyrene and ˜1.5 for glass), making them unsuitable for through-imaging as contemplated herein, whereas hydrogels are available with refractive indices in the range from 1.25 to 1.4, which is close enough to the refractive index of water to allow imaging through the microcarrier to enable visualizing cells on the entire microcarrier surface. For example, gelMA has a refractive index of about 1.34. Additionally, gelMA microcarriers are biodegradable, which improves cell harvesting and manufacturing scalability as compared with, e.g., polystyrene, which is non-degradable and therefore requires a filtration step to harvest cells after expansion. GelMA microcarriers can be synthesized in ways known to those of ordinary skill in the art, e.g., according to a protocol published in R. E. Rogers et al., “A scalable system for generation of mesenchymal stem cells derived from induced pluripotent cells employing bioreactors and degradable microcarriers,” Stem Cells Transl. Med. 10(12), 1650-1665 (2021), which is hereby incorporated herein by reference.

FIG. 3 is an array of example 2D elastic-scattering images of a microcarrier-based cell culture taken with the system of FIG. 2A for twelve different polarization state configurations, illustrating the dependence of image contrast on polarization. The rows of images correspond, in this order from top to bottom, to linear horizontal, linear vertical, right circular, and left circular polarization in the illumination path. The columns of images correspond, in this order from left to right, to linear vertical, linear horizontal, and no polarization in the detection path. The cell culture sample, containing iH-MSCs attached to gelMA microcarriers, was immobilized in agarose within FEP tubing, and imaged by esLSM at 633 nm through a 200 μm thick FEP sheet, using a scanning stage to simulate flow translation. The signal-to-noise ratio (SNR) for each combination of polarization states in the illumination and detection arms was measured as the ratio of cell cytoplasm intensity to the background agarose intensity, and is overlaid onto the respective image. As can be seen, left circular polarized illumination and vertical polarized detection provided the greatest SNR of 12.61. The linear vertical polarized illumination and co-polarized (i.e., also linear vertical) detection state similarly provided high cell SNR. By contrast, the use of linear horizontal polarization for either illumination or detection paths resulted in poor SNR.

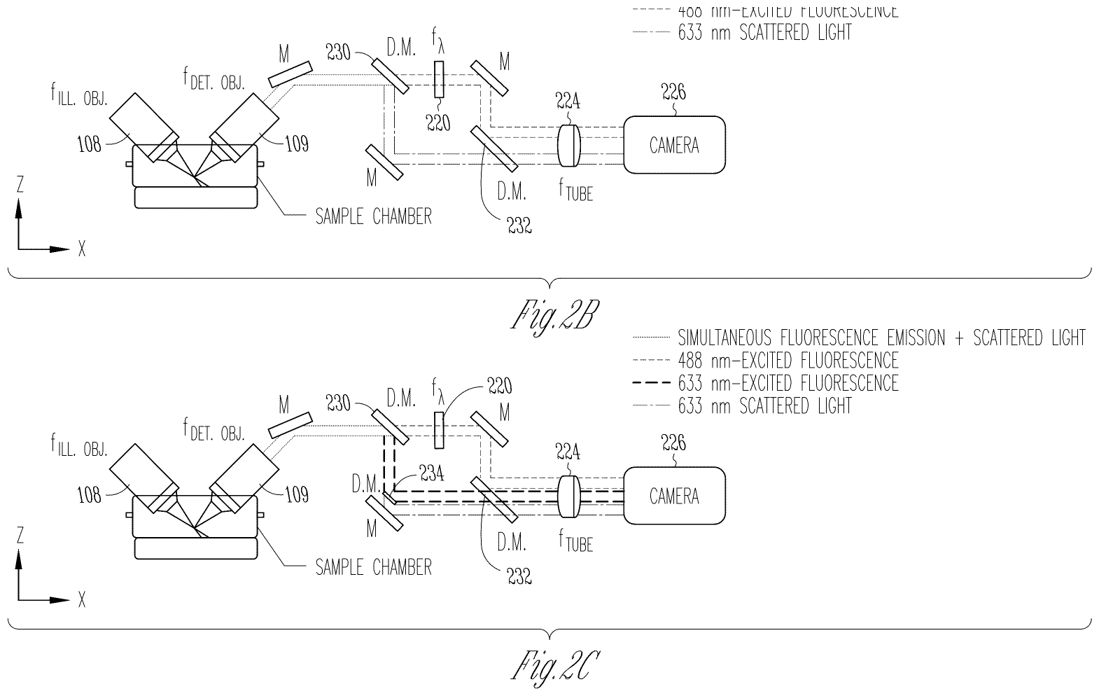

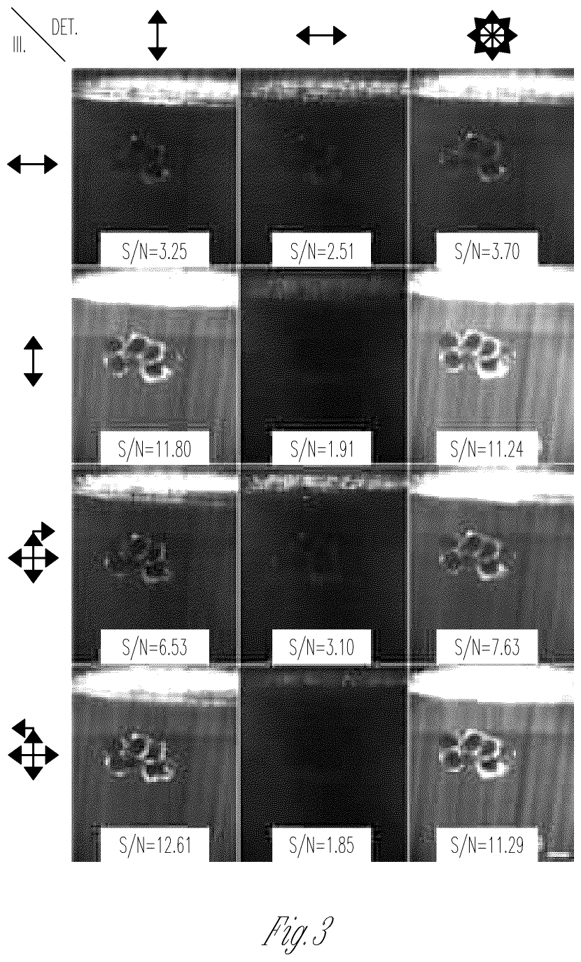

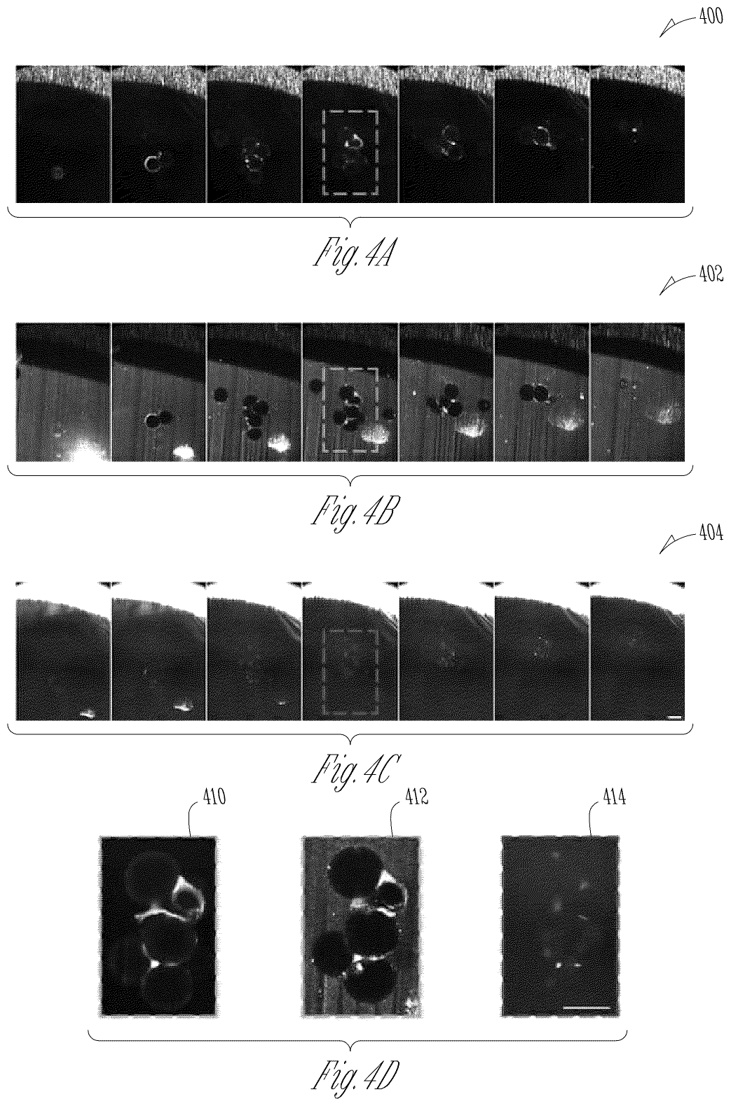

FIGS. 4A-4D show example 2D images of a microcarrier-based cell culture taken with the system of FIG. 2A using various imaging modalities. Like in FIG. 3, the cell culture is an iH-MSC culture on gelMA microcarriers, immobilized in agarose within FEP tubing. The cell cytoplasm and nuclei are fluorescently labeled with CTG and DRAQ-5, respectively. The first row 400 shows a sequence of LSFM images of the CTG fluorescence excited with 488 nm illumination; the second row 402 shows a sequence of esLSM images, acquired at 633 nm using left circular polarized illumination and linear vertical polarized detection; and the third row 404 shows a sequence of LSFM images of the DRAQ-5 fluorescence excited by 633 nm illumination. To simulate flow-based monitoring, the sample was translated during imaging using a scanning stage, and image slices were acquired at a 1-μm spacing between adjacent frames, but the displayed image frames in the rows 400, 402, 404 are about 27 μm apart (that is, only about every 27^{th} frame is shown). In the bottom row, close-ups 410, 412, 414 of the center images of the rows 400, 402, 404, respectively, are shown.

Although the FEP tubing is visible in each image frame of rows 400, 402, 404, it does not cause significant shadowing artifacts that would degrade illumination and image quality. Cellular morphological features can be seen throughout the CTG volume in the images of row 400. In the elastic-scattering images of row 402, the selected polarization configuration minimizes the scattering from the FEP tubing while maximizing cell signal. Using 633 nm illumination and the laser blocking emission filter permits sub-cellular visualization of DRAQ-5-labeled cell nuclei in the images of row 404. As further illustrated in the close-ups 410, 412, 414, LSFM and esLSM provide similar visualization of the iH-MSC morphology throughout the microcarrier aggregate. The shown aggregate includes twelve microcarriers, as enumerated based on the 3D-reconstructed elastic-scattering data, and twenty-three cells, as enumerated using the 3D DRAQ-5 data. The average cell volume was measured to be 4,556 μm³ or 3,677 μm³, as quantified based on CTG and elastic-scattering images, respectively.

FIG. 5 shows an example sequence of 2D elastic-scattering images of a microcarrier-based cell culture sample taken with the system of FIG. 2A using flow-based translation of the sample relative to the light sheet, in accordance with various embodiments. Here, the cell culture sample, once again including iH-MSCs grown on gelMA microcarriers, was formalin-fixed and flown at a volumetric flow rate of about 1000 nL/min through FEP tubing having an inner diameter of about 1 mm (resulting in a flow velocity of about 21 μm/s through the 0.785 mm² cross section of the tubing) Images were taken at a rate of 100 frames per second, again using left circular polarization for illumination and vertical polarization for detection to minimize the scattering in the FEP tubing while preserving the cell signal of interest. FIG. 5 shows images of a populated microcarrier aggregate, which over the course of the image sequence comes in and out of the image plane. As shown more clearly in the close-up 500, highly-scattering cells are discernible on the microcarrier surface and in the aggregate interior. Using intensity-based thresholding, the iH-MSCs can be visually separated from the FEP, the microcarrier, and the water background scattering. Flow-based imaging with elastic-scattering contrast facilitates non-destructive, online monitoring of live cell culture samples.

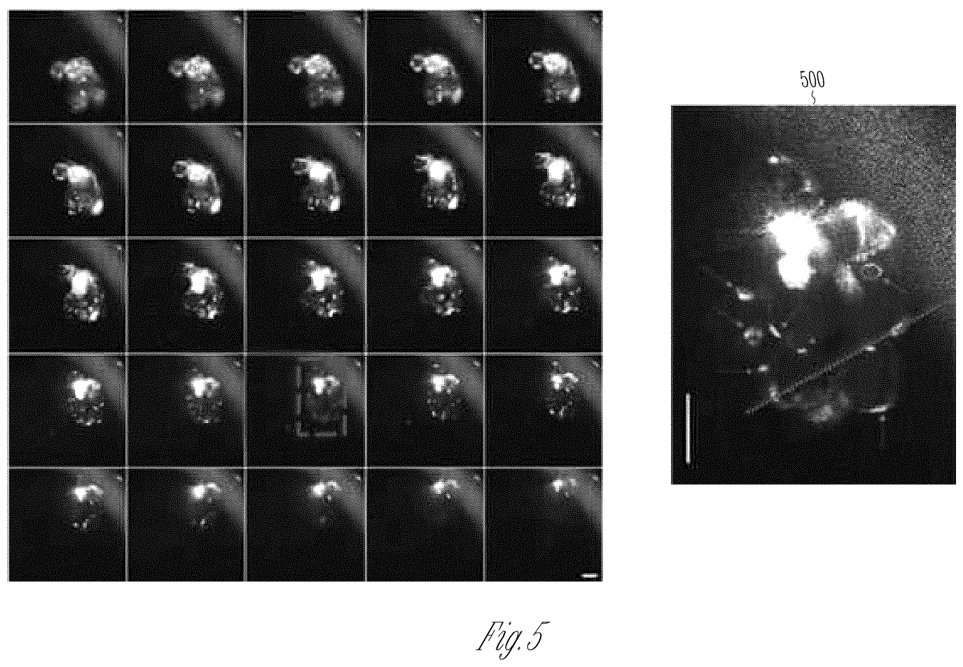

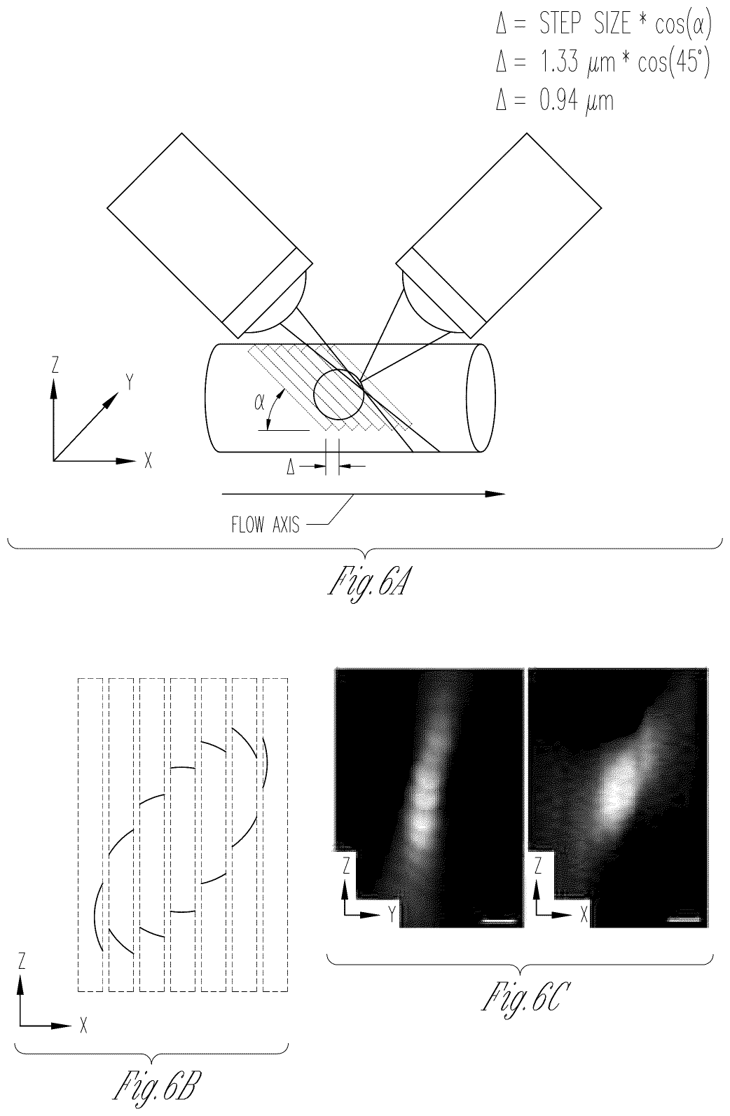

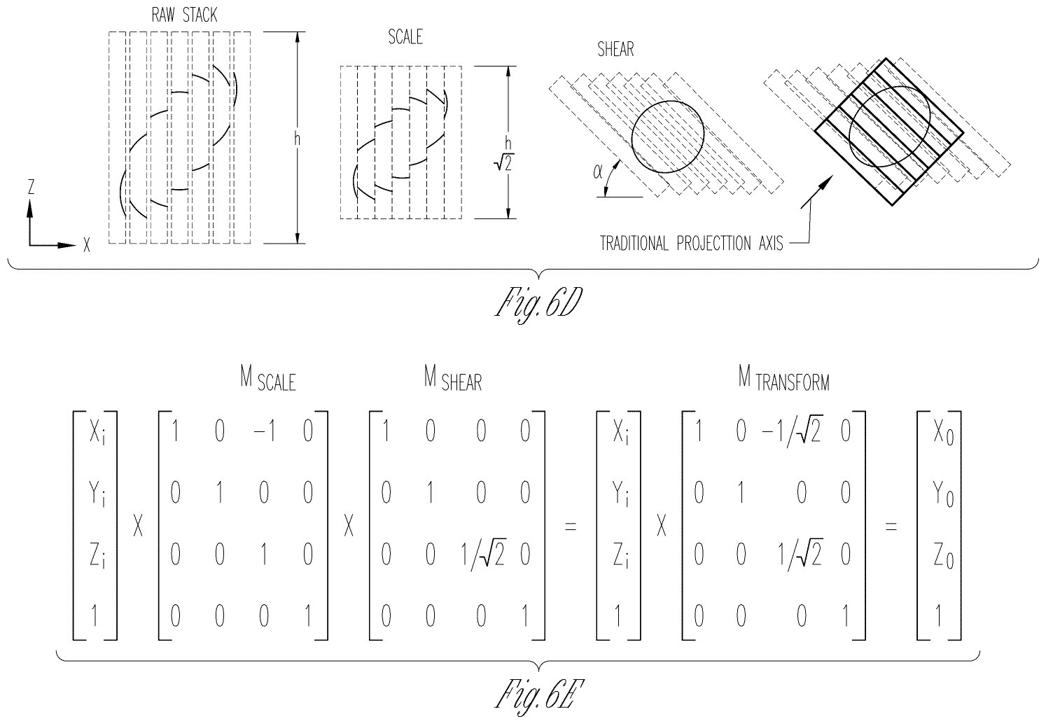

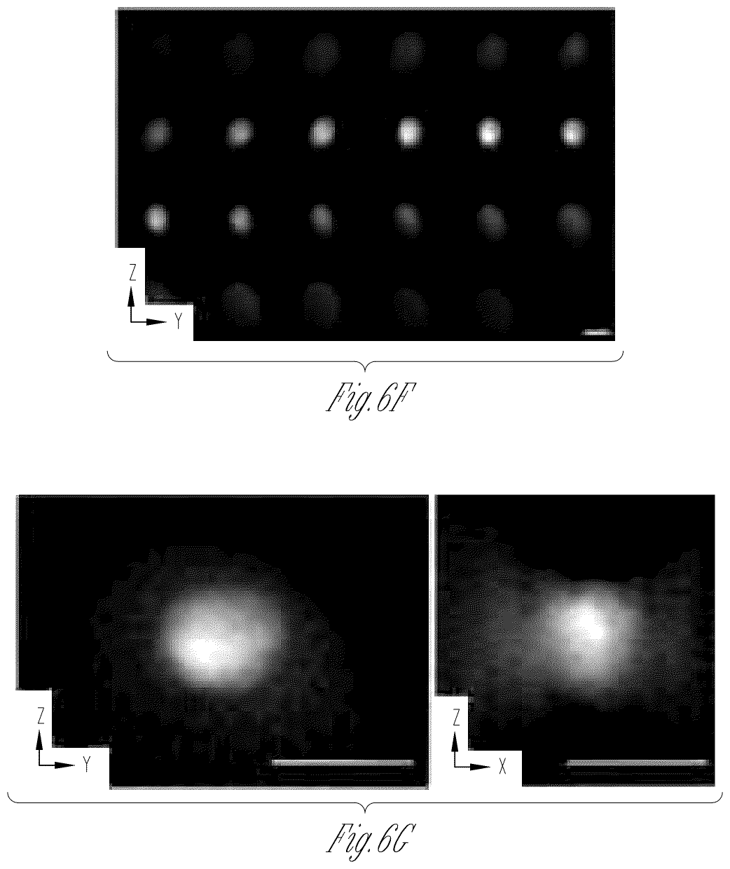

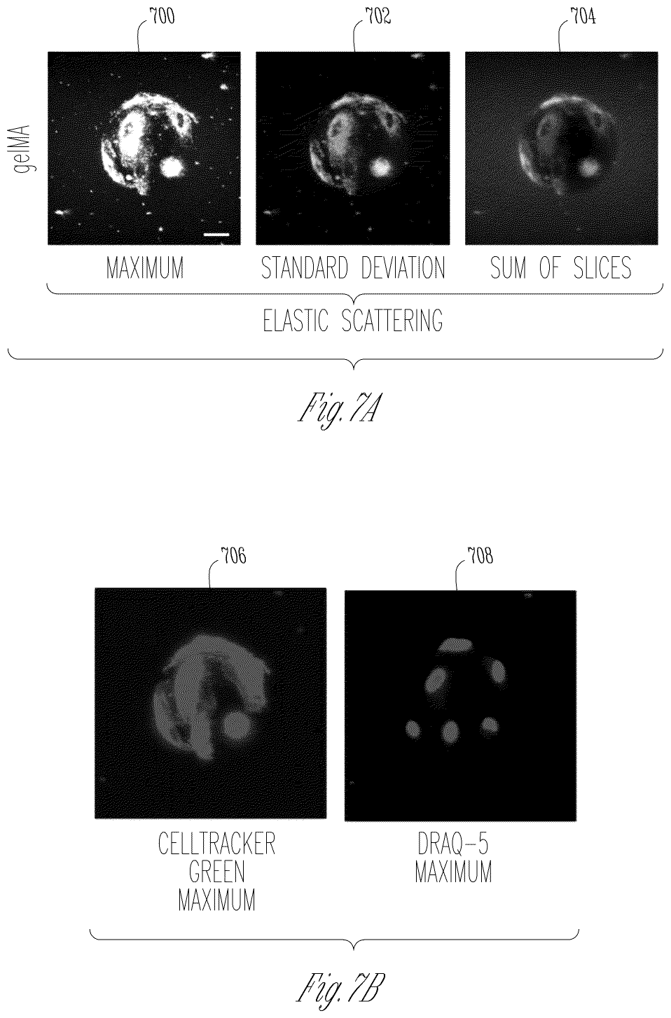

FIGS. 6A-6G illustrate the reconstruction of a 3D volumetric image of a sample from a sequence of 2D images of sample slices, in accordance with various embodiments. The process applies generally to both esLSM and LSFM. For the purpose of illustration, a single fluorescent polystyrene microsphere was used as the sample in this example. As depicted in FIG. 6A, the sample, as it is flown or otherwise translated through the illumination light sheet for optical sectioning, is illuminated along a direction at an angle α relative to the flow axis (or, more generally, axis of sample motion or translation), resulting in a light sheet that encloses the same angle α with the flow axis (or, equivalently, whose normal encloses 90°−α with respect to the flow axis). To facilitate detection of the light emitted from the illuminated sample at a 90° angle relative to the direction of illumination, the light sheet is typically not perpendicular to the direction of flow (i.e., α≠90°). For example, as shown, the light sheet may be tilted by 45° relative to an orientation perpendicular to the flow axis, resulting in both illumination and detection directions at 45° with respect to the flow axis. Due to this oblique imaging geometry, the thickness of each sample slice d_{slice} is reduced to 1/\sqrt{2} times the axial step size v_{flow}/r_{acq} at which images are acquired (where v_{flow} is the flow velocity, e.g., in μm/s, and r_{acq} is the image acquisition rate, e.g., in 1/s), and the imaged sample slices appear shifted relative to each other, or skewed, in a stack of raw images. FIG. 6B shows this skew diagrammatically for a projection of the image data into the x-z plane, x being the direction of flow, and FIG. 6C shows the skew with raw image data projected into the x-z and y-z planes using standard image visualization software that does not pre-process the image data.

As part of reconstructing a 3D image of the sample, the raw volumetric image data can be de-skewed, e.g., by linearly displacing pixels a pre-set number of frames determined by the illumination angle α and the axial step size, or by an affine transformation to scale and shear the original voxels to the correct dimensions and locations. FIG. 6D schematically illustrates an affine transformation performed on the stack of raw images to de-skew the volumetric image data, and FIG. 6E shows the corresponding matrix operations effecting this transformation. A traditional en-face projection can be acquired by rotating the imaged object. FIG. 6F is a montage of image slices of the sample (i.e., in this case, a microcarrier sphere), showing a traditional en-face view along the projection axis indicated in FIG. 6D. FIG. 6G shows maximum intensity projections of the data of FIG. 6F into the y-z and x-z planes.

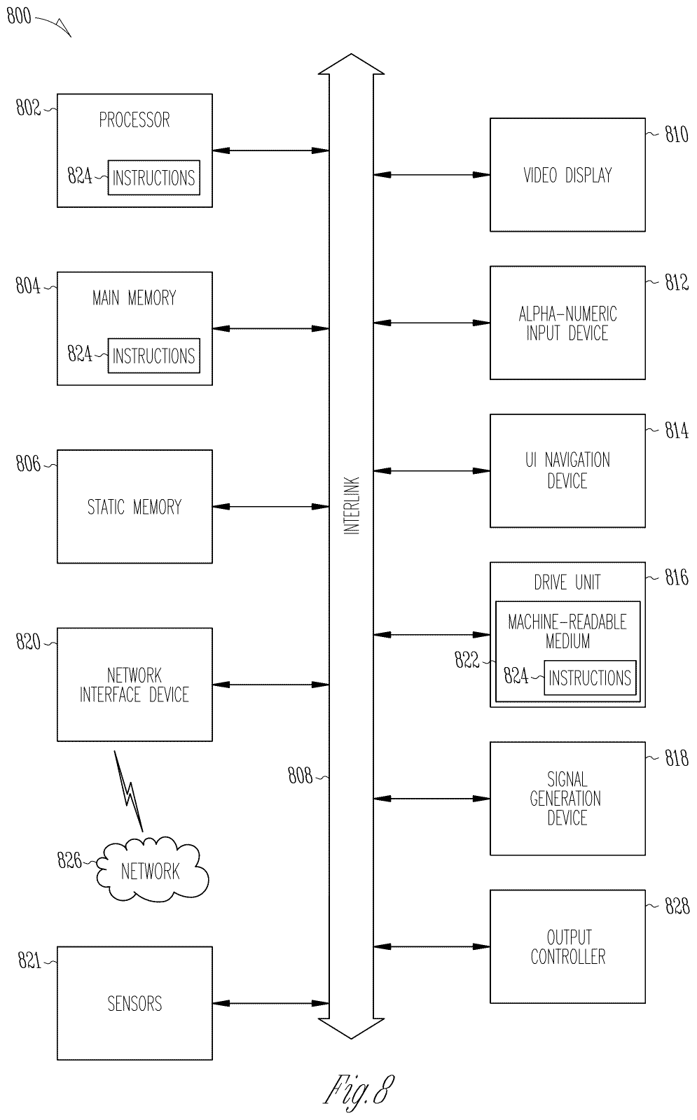

FIGS. 7A and 7B are volumetric projections of example 3D volumetric images of iH-MSC cultured on a gel-MA microcarrier, as reconstructed from esLSM data and LSFM data, respectively. In FIG. 7A, the 3D esLSM image is visualized in two dimensions with maximum intensity projection 700, standard deviation projection 702, and sum of slices projection 704. As these projections show, esLSM allows sub-cellular visualization of cells along the entire 3D surface of the microcarrier. The standard deviation and sum of slices projections even permit simultaneous visualization of the microcarrier, cells, and surrounding agarose. The LSFM images 706, 708 in FIG. 7B show, for comparison, the CTG-labeled cytoplasm and the DRAQ-5-labeled nuclei of the cells, respectively, illustrating good correlation between the elastic-scattering and fluorescence data.

In accordance with various embodiments, the 3D volumetric image of a cell culture sample is further analyzed, by suitable computer-implemented algorithms, to determine one or more cell culture parameters, such as, without limitation, the number or total volume of cells in the sample, one or more cell size parameters such as average size, one or more cell morphology parameters such as an average shape, or for microcarrier-based cell cultures, the number of microcarriers and number of cells per microcarrier.

In some embodiments, image analysis involves rules-based processing using, e.g., intensity and size thresholds. An example software product suitable for such analysis is Imaris AI Microscopy Image Analysis Software by Oxford Instruments. Imaris provides a “spot” function for identifying small, point-like objects within a volumetric image, and a “surface” function for outlining larger, continuous structures. By selectively using these functions, in conjunction with setting intensity and size filters, both fluorescence and elastic-scattering 3D images can be processed to determine cell culture parameters. For direct cell enumeration, for instance, the “spot” function may be used on DRAQ-5 fluorescence data with an object size filter of 10 μm for automatic detection of cell nuclei, followed by a manual high-pass intensity threshold to further segment out cell debris and ultimately enumerate only cell nuclei. For quantification of cell volume, the “surface” function may be used on the CTG fluorescence data with a manual high-pass intensity threshold and size filter to remove cell debris from quantification. To enumerate microcarriers, the “surface” function may be used on the CTG data with a low-pass intensity-based threshold to create solid objects, and then, the “spot” function with a suitable size filter, e.g., a 90 μm size filter for microcarriers around 120 μm in diameter, can automatically count individual microcarriers. When processing elastic-scattering data, the “surface” function may be used for image segmentation, e.g., to separate cells and microcarriers from each other and the surrounding medium. For cell volume quantification, a high-pass intensity threshold and a high-pass size filter may be used. For microcarrier enumeration, a low-pass intensity threshold and 90 μm size filter may be used. Once individual cells are identified, their sizes and overall shape (e.g., in terms of ellipticity) can also be measured in the image.

In some embodiments, a machine-learning model is employed to “predict” cell culture parameters from the 3D image data. The machine-learning model may, for instance, be a neural network model, e.g., including one or more convolutional neural network layers, as are common in image processing. The model can be trained in a supervised manner based on training data that includes, for each of a (usually large) number of cell culture samples, a 3D image of the cell culture sample, labeled with an associated “ground-truth” cell culture parameter (or parameters) of the sample. The ground-truth cell culture parameters, or labels, may be determined, e.g., by rules-based processing of the image as described above, or by some other method. Model training involves iteratively feeding 3D images as input into the model to compute corresponding output predictions of the cell culture parameters, comparing the predicted cell culture parameters computed by the model against the ground-truth cell culture parameters, and updating adjustable parameters, or weights, of the model to reduce the discrepancy between model outputs and ground truth. This iterative optimization, or “learning,” of the weights of the model can be performed, e.g., using the well-known back-propagation of errors algorithm. In some embodiments, correlated elastic-scattering and fluorescence data may be used as training data to train the model in a supervised manner to determine cell culture parameters from elastic-scattering images. That is, for a given pair of an elastic-scattering 3D image and a fluorescence 3D image of the same sample, the fluorescence data may be analyzed, e.g., using the rules-based processing described above, to determine cell culture parameters, which can then serve as labels of the associated elastic-scattering data.

FIG. 8 is a block diagram of an example machine 800 suitable for controlling the operation of the systems of FIGS. 1 and 2 and/or implementing functionality for processing and analyzing the elastic-scattering and/or fluorescence images, in accordance with various embodiments. For example, the machine 800 may implement the processing system 120 of FIG. 1, and/or the image processing and analysis system discussed throughout this application. In alternative embodiments, the machine 800 may operate as a standalone device or may be connected (e.g., networked) to other machines. In a networked deployment, the machine 800 may operate in the capacity of a server machine, a client machine, or both in server-client network environments. In an example, the machine 800 may act as a peer machine in peer-to-peer (P2P) (or other distributed) network environment. The machine 800 may be a personal computer (PC), a tablet PC, a set-top box (STB), a personal digital assistant (PDA), a mobile telephone, a smartphone, a web appliance, a server computer, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Machine (e.g., computer system) 800 may include a hardware processor 802 (e.g., a central processing unit (CPU), a graphics processing unit (GPU), a hardware processor core, or any combination thereof), a main memory 804 and a static memory 806, some or all of which may communicate with each other via an interlink (e.g., bus) 808. The machine 800 may further include a display unit 810, an alphanumeric input device 812 (e.g., a keyboard), and a user interface (UI) navigation device 814 (e.g., a mouse). In an example, the display unit 810, input device 812 and UI navigation device 814 may be a touch screen display. The machine 800 may additionally include a storage device (e.g., drive unit) 816, a signal generation device 818 (e.g., a speaker), a network interface device 820, and one or more sensors 821, such as, e.g., the flow sensors described with reference to FIG. 1. The machine 800 may include an output controller 828, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 816 may include a machine-readable medium 822 on which are stored one or more sets of data structures or instructions 824 (e.g., software) embodying or utilized by any one or more of the techniques or functions described herein. The instructions 824 may also reside, completely or at least partially, within the main memory 804, within static memory 806, or within the hardware processor 802 during execution thereof by the machine 800. In an example, one or any combination of the hardware processor 802, the main memory 804, the static memory 806, or the storage device 816 may constitute machine-readable media. While the machine-readable medium 822 is illustrated as a single medium, the term “machine-readable medium” may include a single medium or multiple media configured to store the one or more instructions 824 and associated data (e.g., image data).

In general, the term “machine-readable medium” may include any medium that is capable of storing, encoding, or carrying instructions for execution by the machine 800 and that cause the machine 800 to perform any one or more of the techniques of the present disclosure, or that is capable of storing, encoding or carrying data structures (including, e.g., image data) used by or associated with such instructions. Non-limiting machine-readable medium examples may include solid-state memories, and optical and magnetic media. Specific examples of machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; Random Access Memory (RAM); Solid State Drives (SSD); and CD-ROM and DVD-ROM disks. In some examples, machine-readable media may include non-transitory machine readable media. In some examples, machine-readable media may include machine-readable media that are not a transitory propagating signal.

The instructions 824 may further be transmitted or received over a communications network 826 using a transmission medium via the network interface device 820. The machine 800 may communicate with one or more other machines utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, a Long Term Evolution (LTE) family of standards, a Universal Mobile Telecommunications System (UMTS) family of standards, peer-to-peer (P2P) networks, among others. In an example, the network interface device 820 may include one or more physical jacks (e.g., Ethernet, coaxial, or phone jacks) or one or more antennas to connect to the communications network 826. In an example, the network interface device 820 may include a plurality of antennas to wirelessly communicate using at least one of single-input multiple-output (SIMO), multiple-input multiple-output (MIMO), or multiple-input single-output (MISO) techniques. In some examples, the network interface device 820 may wirelessly communicate using Multiple User MIMO techniques.

Although embodiments have been described with reference to specific example embodiments, it will be evident that various modifications and changes may be made to these embodiments without departing from the broader scope of the invention. Accordingly, the specification and drawings are to be regarded in an illustrative rather than a restrictive sense.

1. A system for monitoring three-dimensional (3D) cell culture in suspension, the system comprising:
a fluidic sampling system comprising a sample chamber and configured to flow a sample of the 3D cell culture through the sample chamber;
a light sheet microscopy system configured to generate an illumination light sheet at a location inside the sample chamber and to acquire a two-dimensional (2D) image of a sample slice within the illumination light sheet; and
an image processing and analysis system configured to process a sequence of 2D images acquired by the light sheet microscopy system as the sample is flown through the illumination light sheet to reconstruct a 3D volumetric image of the sample.

2. The system of claim 1, wherein the 3D cell culture is grown in a bioreactor, and wherein the fluidic sampling system is a closed-loop system further comprising tubing to flow the sample from the bioreactor to the sample chamber and back to the bioreactor.

3. The system of claim 1, wherein:
the fluidic sampling system further comprises a pump operable to control a flow rate of the sample through the sample chamber;
the light sheet microscopy system comprises a camera having a controllable image acquisition rate for acquiring the sequence of 2D images; and
the system further comprises a controller configured to control the flow rate and the image acquisition rate to achieve a predetermined sample slice thickness.

4. The system of claim 1, wherein a wall of the sample chamber comprises a polymer having a refractive index between 1.32 and 1.35.

5. The system of claim 4, wherein the polymer is fluorinated ethylene propylene (FEP).

6. The system of claim 1, wherein the light sheet microscopy system is selectively configurable for both elastic-scattering contrast and fluorescence contrast of the 2D image of the sample slice.

7. The system of claim 6, wherein the light sheet microscopy system comprises a first light source configured to create a first beam of illumination light having a first wavelength, a second light source configured to create a second beam of illumination light having a second wavelength different from the first wavelength, and a dichroic mirror configured to coalign the first beam and the second beam.

8. The system of claim 7, wherein the light sheet microscopy system is configurable for elastic-scattering contrast at the first wavelength, and for fluorescence contrast using an excitation wavelength equal to the second wavelength.

9. The system of claim 1, wherein the light sheet microscopy system comprises an illumination objective lens configured to focus illumination light into the sample chamber and a detection objective lens configured to collect light emitted from within the illumination light sheet, wherein refractive indices along an optical path between the illumination and detection objective lenses are substantially matched.

10. The system of claim 1, wherein the light sheet microscopy system comprises a polarizer in an illumination path and a polarization analyzer in a detection path of the light sheet microscopy system.

11. The system of claim 10, wherein the polarizer is configured for one of left circular polarized illumination or vertical illumination, and wherein the polarization analyzer is configured for vertical polarized detection.

12. The system of claim 1, wherein the image processing and analysis system is further configured to analyze the 3D volumetric image to determine one or more cell culture parameters, the one or more cell culture parameters comprising at least one of: a number of microcarriers in the sample, a total cell volume in the sample, a number of cells in the sample, an average number of cells per microcarrier, a cell morphology parameter, or a cell size parameter.

13. A method for on-line, label-free monitoring of three-dimensional (3D) cell culture grown in suspension a bioreactor, the method comprising:
flowing a sample of the 3D cell culture from the bioreactor through a sample chamber and back to the bioreactor;
focusing illumination light to form a light sheet inside the sample chamber and, as the sample is flown through the light sheet, detecting elastically scattered light from the sample to acquire a sequence of two-dimensional (2D) images of respective slices of the sample; and
computationally reconstructing a 3D volumetric image of the sample from the sequence of 2D images.

14. The method of claim 13, wherein the 3D cell culture is a microcarrier-based cell culture.

15. The method of claim 14, wherein the microcarrier-based cell culture comprises cells grown on hydrogel microcarriers having a refractive index between 1.25 and 1.4.

16. The method of claim 15, wherein the hydrogel microcarriers comprise gelatin methacryloyl (gelMA).

17. The method of claim 13, further comprising:
computationally processing the 3D volumetric image to determine one or more cell culture parameters.

18. The method of claim 17, wherein the one or more cell culture parameters comprise at least one of: a number of microcarriers in the sample, a total cell volume in the sample, a number of cells in the sample, an average number of cells per microcarrier, a cell morphology parameter, or a cell size parameter.

19. The method of claim 13, further comprising controlling a polarization of the illumination light to be left circular or vertical prior to focusing, and passing the elastically scattered light through a vertical polarization analyzer prior to detection.

20. The method of claim 13, coordinate a flow velocity of the sample through the sample chamber with an image acquisition rate associated with the sequence of 2D images to achieve a predetermined thickness of slices of the sample.
